(19) United States
(12) Patent Application Publication
(10) Pub. No.: US 2005/0148872 A1
(43) Pub. Date: Jul. 7, 2005

(54) Apparatus and method for synthetic focus ultrasonic imaging

(76) Inventor: William D. Richard, Ballwin, MO (US)

Correspondence Address: POLSTER, LIEDER, WOODRUFF & LUCCHESI, 12412 POWERSCOURT DRIVE SUITE 200, ST. LOUIS, MO, 63131-3615, US

(21) Appl. No.: 11/024,168

(22) Filed: Dec. 22, 2004

Related U.S. Application Data
(60) Provisional application No. 60/532,086, filed on Dec. 23, 2003.

(51) Int. Cl. A61B 8/00

(52) U.S. Cl. 600/443

(57) ABSTRACT

An ultrasonic imaging apparatus configured to generate synthetic focus ultrasonic images in real-time in a first operation mode, and which is configured to pulse multiple transducers with accurate time delays between each pulse to form images using conventional sum beam-forming and/or focus-and-steer techniques in a second mode, providing a “dual-use” system capable of forming both synthetic focus images and beam-formed images using the same transducer array.

### CROSS-REFERENCE TO RELATED APPLICATIONS

The present application is related to, and claims priority from, U.S. Provisional Patent Application No. 60/532,086 filed on Dec. 23, 2003, herein incorporated by reference.

### STATEMENT REGARDING FEDERALLY SPONSORED RESEARCH

Not Applicable.

### BACKGROUND OF THE INVENTION

The present invention is related generally to ultrasonic imaging systems, and in particular to an ultrasonic imaging system utilizing a scalable architecture configured to generate real-time synthetic focus images.

Ultrasonic imaging systems are used in a wide range of applications ranging from fetal imaging to non-destructive evaluation. In developed countries, almost every fetus is imaged using ultrasonic imaging to monitor growth and development and to evaluate fetal health. Medical ultrasound systems are used in a wide variety of medical applications, ranging from cardiac evaluation to intra-operative neurosurgery for tumor location to breast cancer screening. Some example of non-medical uses of ultrasonic imaging systems include the location of faults in structures such as steel beams and aircraft wings, seismic imaging for mineral exploration, and synthetic-aperture radar for defense and commercial applications.

Unlike magnetic resonance imaging (MRI) or computer tomography (CT) systems, ultrasonic imaging systems provide real-time images. The generation of real-time images renders ultrasonic imaging systems attractive for many applications. In addition, when compared to MRI or CT systems, ultrasonic imaging systems are much lower in cost, and, as such, are the preferred method for imaging when cost is a concern (as it is in screening applications where large populations need to be imaged). Ultrasonic imaging uses non-ionizing radiation, unlike CT imaging systems, and is thus considered to have far fewer risks, especially when used over a period of many years as a screening method.

Traditional array-based ultrasonic imaging systems use a “focus and steer” method for forming images. In the “focus and steer” method, an ultrasonic beam is focused to transmit and receive at selected image points or pixels. For real-time operation, typically about 100 pixels are in focus. Producing a focus and steer image which is in exactly in-focus at each image pixel requires a data acquisition time which is the product of the number of image pixels and the round-trip time of the ultrasonic wave.

For acceptable image sizes, the data acquisition time using the “focus and steer” method in an ultrasonic imaging system is so large that it is impractical to form real-time images which are in focus at each image pixel. Hence, in a system utilizing “focus and steer” methods, absolute focus of each pixel in the image is compromised in order to achieve real-time frame rates.

An alternative to “focus and steer” methods in ultrasonic imaging, known as synthetic focus imaging, uses a complete dataset of image data. All transmitter-receiver array element pairs are used to acquire ultrasonic backscatter data. The data acquisition time for a synthetic focus imaging approach to the generation of ultrasonic images, which are in-focus at each pixel, is short enough to support real-time imaging for acceptable image sizes, e.g., 512 by 512 pixels. The computation requirements, however, for synthetic focus imaging are very large.

Synthetic focus imaging offers the possibility of providing for early detection and staging of cancers, especially for static, easy to insonify glands like breast and prostate tissue. Cancers in these tissues are among the leading causes of new cancer cases. Ultrasonic imaging is currently utilized to detect and stage these cancers, but the systems are limited by resolution and contrast capabilities. Synthetic focus imaging systems separate data acquisition from image formation and can provide in-focus information at every image pixel. This permits image contrast to be easily adjusted to compensate for various properties of the tissues being examined. However, current synthetic focus imaging systems have a relatively long image-formation time. This is due to the fact that the synthetic-focus image acquisition time is proportional to the number of ultrasonic transducers in the transmit/receive array. Conversely, the time required for image formation using convention focus-and-steer configurations is proportional to the number of focal points in the image. Therefore, since the number of pixels in an image is typically orders of magnitude greater than the number of transducers, acquiring an in-focus image with conventional systems is impractical.

In synthetic-focus imaging systems, the following computation is required to calculate a single pixel p(i,j) using data from an N element transducer array (N sources and N sensors), where TOF(i, j, m, n) is the time-of-flight contribution to pixel p(i,j) from source m and sensor n:

$$p ( i , j ) = {\sum }_{m = 1}^{m = N} {\sum }_{n = 1}^{n = N} f ( TOF ( i , j , m , n ) )$$

The time required to generate an image can be broken down into the following principal tasks: (1), the time-of-flight (TOF) calculation; (2), the retrieval of backscattered signals from memory; and (3), the summation of backscattered values to define each pixel value p(i,j).

Linear array and phased array ultrasonic imaging systems that use the traditional “focus and steer” method for forming real-time images are common. U.S. Pat. No. 6,719,693 B2 to Richard for “Apparatus and System For Real-Time Synthetic Focus Ultrasonic Imaging” provides a real-time synthetic focus ultrasonic imaging system developed which captures images large enough to be used for diagnostic purposes. Alternatively, synthetic focus ultrasound images have been formed using data acquisition hardware and off-line computation engines, including single processor and multi-processor computer systems. However, none of these systems is capable of switching between synthetic-focus image acquisition and a focus-and-steer image acquisition mode to select an imaging processes which is most suitable for the current application in terms of image acquisition speed and image resolution.

A method for computing, in real-time, the time-of-flight surfaces required to form synthetic focus images is described by S. R. Broadstone and R. M. Arthur in “An Approach To Real-Time Reflection Tomography Using The Complete Dataset”, in proceedings 1986 Ultrasonics Symposium, Vol. 86CH2375-4, pp. 829-831, IEEE Press, 1986, (the Broadstone and Arthur reference). The Broadstone and Arthur reference further describes an integrated circuit implementation of the disclosed method. In the integrated circuit design, one time-of-flight calculator is required for each transmit/receive transducer pair in a massively parallel ultrasonic imaging system in order to form real-time synthetic focus images. However, the Broadstone and Arthur reference does not provide a complete, realizable architecture for a synthetic-focus imaging system capable of being constructed using currently available integrated circuit components or technologies.

Current ultrasonic imaging systems which utilize the “complete data set” for image formation require large numbers of components per channel for data storage and image generation. These hardware requirements would be exacerbated in the implementation of a real-time imaging system, and, for this reason, none have been constructed.

Accordingly, there is a need for a real-time synthetic-focus ultrasonic imaging system having the ability to pulse multiple transducers simultaneously with accurate time delays between each pulse to form images using conventional sum beam-forming and/or focus-and-steer techniques, permitting the development of a “dual-use” system capable of forming both synthetic focus images and images based on the more conventional approach of sum beam-forming. A dual-use system will have more utility in a clinical setting since it can support the mode most appropriate for the imaging task at hand.

### BRIEF SUMMARY OF THE INVENTION

Briefly stated, the present invention provides an apparatus for use in an ultrasonic imaging system adapted for generating both synthetic focus images and sum beam-forming/focus-and-steer images. The apparatus employs a parallel processing integrated circuit architecture which is scalable and can be employed to construct real-time synthetic focus ultrasonic imaging systems having a large number of transducer array elements. The apparatus provides the ability to quickly and iteratively generate candidate final images based on a single complete dataset of image pixels, and facilitates the development of optimization techniques for accurate speed of sound extraction to enable the generation of synthetic focus images. In addition, the apparatus provides the ability to pulse multiple transducers with accurate time delays between each pulse, permitting the formation of images using sum beam forming/focus-and-steer techniques. This dual-use apparatus is configured to provide multiple modes of operation, selected for the appropriate imaging task at hand.

A method of the present invention propagates ultrasonic energy towards a sample object and receives the reflected echoes. The received echoes are digitized and stored, and subsequently utilized with one or more coefficients to generate an ultrasonic image of the sample object. The method includes the step of selecting between at least two modes of image formation, real-time synthetic focus imaging mode in which individual transducers are pulsed discretely, and the reflected signals received at all transducers, and a sum beam-forming/focus-and-steer imaging mode in which multiple transducers are pulsed at selected time delays and in selected sequences. By altering the delay timing, pulse sequence, and coefficients, adaptive image generation from the complete set of image pixel data facilitates the generation of subsequent images having varied focus or contrast from the initial image.

The foregoing and other objects, features, and advantages of the invention as well as presently preferred embodiments thereof will become more apparent from the reading of the following description in connection with the accompanying drawings.

### BRIEF DESCRIPTION OF THE SEVERAL VIEWS OF THE DRAWINGS

In the accompanying drawings which form part of the specification:

FIG. 1 is a prior art scalable architecture for real-time synthetic focus ultrasonic imaging;

FIG. 2 is an illustration of the ultrasonic wavefronts created by pulsing a singe transducer of the architecture of FIG. 1 when forming synthetic focus ultrasonic images;

FIG. 3 is a prior art internal architecture of the “F” field programmable gate array component of FIG. 1;

FIG. 4 is an exemplary illustration of a hardware embodiment of the present invention including a motherboard and daughter boards;

FIG. 5 is a block diagram representation of one daughterboard of FIG. 4; and

FIG. 6 is an illustration of an ultrasonic wave front created by pulsing multiple transducers at time-delayed intervals.

Corresponding reference numerals indicate corresponding parts throughout the several figures of the drawings.

### DESCRIPTION OF THE PREFERRED EMBODIMENT

The following detailed description illustrates the invention by way of example and not by way of limitation. The description clearly enables one skilled in the art to make and use the invention, describes several embodiments, adaptations, variations, alternatives, and uses of the invention, including what is presently believed to be the best mode of carrying out the invention.

A scalable architecture for forming real-time synthetic focus images is described in U.S. Pat. No. 6,719,693 B2 to Richard, entitled “Apparatus and System for Real-Time Synthetic Focus Imaging”, herein incorporated by reference. By adding time delay circuits to provide the ability to pulse multiple transducers with accurate time delays between each pulse, the scalable architecture described therein can be used to additionally form images using a sum beam-forming/focus-and-steer imaging technique. This addition enables a “dual-use” system capable of forming both synthetic focus images and images based on sum beam-forming. A dual-use system provides increased utility in a clinical setting since it can support the mode most appropriate for the imaging task at hand.

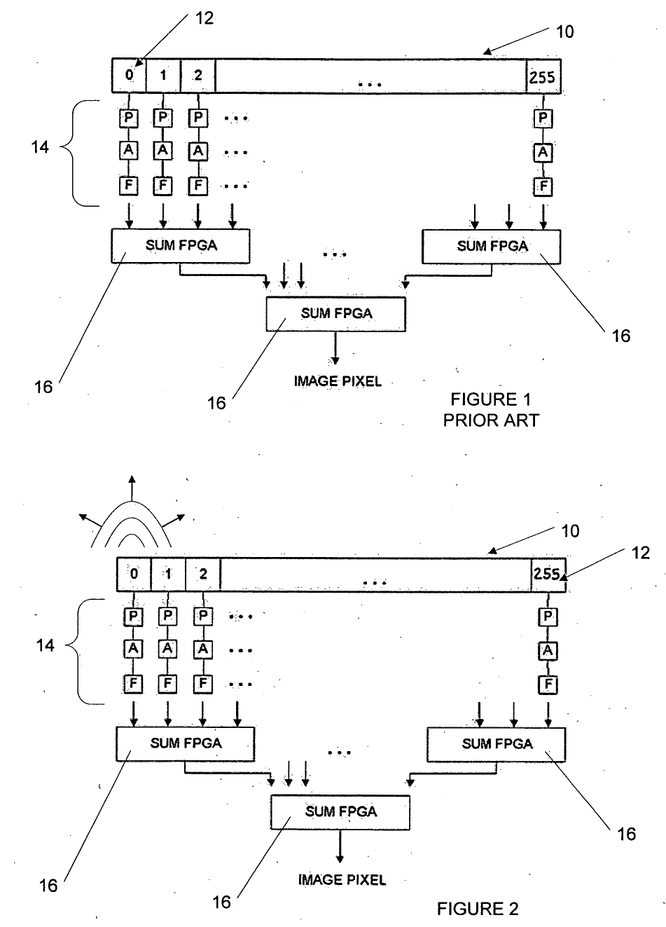

Turning to FIG. 1, a 256-element one-dimensional ultrasonic transducer array of a scalable architecture, such as that founding U.S. Pat. No. 6,719,693 B2 for forming real-time two-dimensional synthetic focus images is shown generally at 10. Each element 12 of the array 10 includes a set of processing circuits, indicated generally at 14. The first processing circuit in each set 14, indicated as “P”, consists of an ultrasonic transmitter configured to propagate a pulse of ultrasonic energy toward a sample undergoing testing or imaging, and an ultrasonic receiver configured to receive ultrasonic return echoes reflected from the sample. Coupling to each pulser/receiver “P” is an analog-to-digital converter, designated as “A”. The output from each analog-to-digital converter “A” is stored in an associated image generation processor, preferably a field programmable gate array (FPGA) “F”. The preferred embodiment of the architecture uses one FPGA (or ASIC) associated with each array transducer element (or group of elements) to form real-time two-dimensional images.

In order to form synthetic focus ultrasound images, each transducer is pulsed individually and the return signals received at all transducers recorded in a block memories inside the associated FPGAs (or ASICs). FIG. 2 illustrates the outward propagation of spherical ultrasonic wavefronts generated when a single transducer is pulsed.

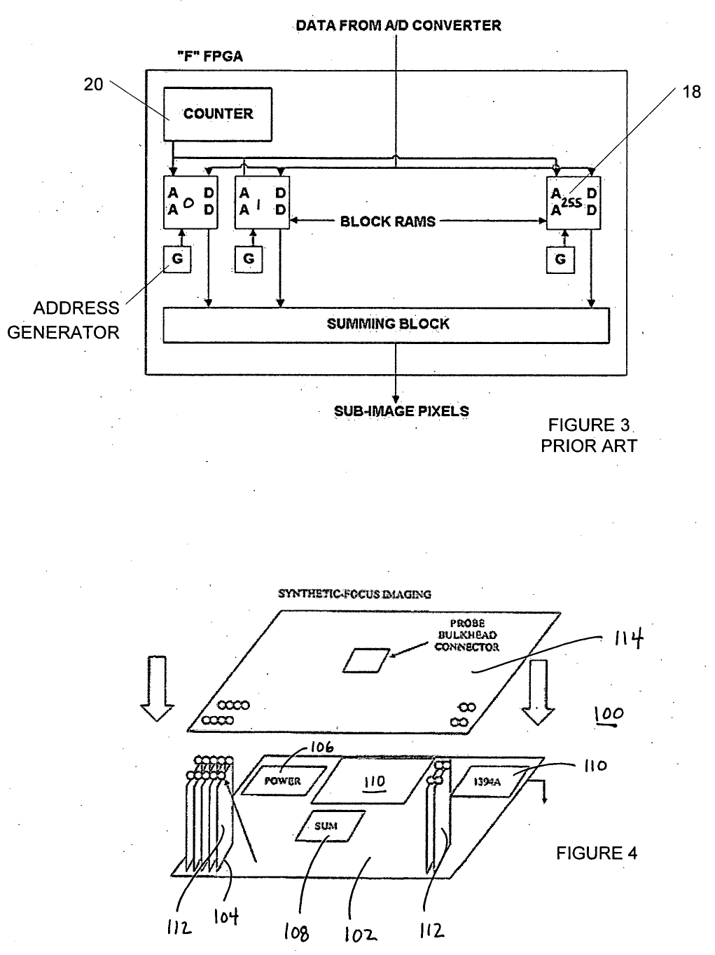

The internal architecture of each “F” FPGA is shown in FIG. 3. In this embodiment, each “F” FPGA will use 256 on-chip dual-port block random-access memory (RAM) modules 18 to store the 256 digitized return echoes from associated transducer array element P. When element “0” in the array is pulsed, each “F” FPGA stores the digitized return echo from an associated transducer element P in internal block RAM “0”. When element “1” in the array is pulsed, each “F” FPGA stores the digitized return echo from an associated element in block RAM “1”, etc. Each dual-port block RAM 18 is filled, in turn, as the elements P of the transducer array are pulsed. Data from the external analog-to-digital converter “A” in each set 14 drives the data inputs on one side of each dual-port block RAM 18. The required address is generated using a simple counter 20 that is reset to zero at the beginning of each pulse/receive/acquisition cycle by a global controller. This architecture allows the partial dataset associated with a particular receiver to be stored in the associated FPGA ‘F” and the complete dataset to be stored in the entire set of FPGAs.

A “G” block address generator is associated with each dual-port block RAM 18 inside the “F” FPGA. The dual-port block RAMs 18 are accessed in parallel during image generation so that the sub-image associated with the data inside each FPGA ‘F” can be calculated in real time (one sub-image pixel can be calculated on each access cycle/clock cycle). The required addresses are calculated by the “G” block address generators constructed using the logic available inside the “F” FPGA. In the example used here, 256 independent address calculators “G” are implemented inside each FPGA ‘F” using known implementation techniques.

After a complete dataset has been acquired, a global controller initiates an image generation cycle. This resets each “G” address generator to, for example, the upper left corner pixel of the sub-image, and each “G” address generator outputs the associated address for its block RAM 18. All block RAMs 18 are accessed in parallel, and, in one RAM access cycle, all of the samples required to form the first sub-image pixel are read from the dual port block RAMs 18 in all of the FPGAs “F”. An adder tree/summing network is used to add the samples to form the first pixel in the sub-image inside each “F” FPGA. This summation may need to be pipelined, but this is easily accomplished inside the “F” FPGA. The output of the summing network is feed out of the FPGA ‘F” so that it can be added to the other sub-images that are generated in parallel from the other “F” FPGAs. In the embodiment shown in FIG. 1, a set of Sum FPGAs 16 simply sum the sub-images generated by the “F” FPGAs to produce the final real-time image. This summation is done in a tree fashion, and more levels of summation may be required than the number shown FIG. 1. Physical pin limitations on the particular configuration of FPGA logic circuits used will determine the number of levels required.

The “G” address generators are designed to output the next associated sample address each time they are clocked, and the sub-image pixels are generated one-per-clock-cycle. Using a 100 MHz clock, 512×512 pixel images can be formed at least at the rate of 38 frames/second, if acquisition time is ignored. The “G” address generators can be implemented using conventional techniques, such as described in U.S. Pat. No. 6,719,693 B2. As integration levels increase, it will be possible to directly calculate the required time of flight inside each “G” address generator and still produce one address per clock cycle (or one address every N clock cycles if a slightly lower frame rate is acceptable). As integration levels increase, it is further anticipated that required address values will be stored in a table. The architecture described here can use any of these possible forms for the “G” address generator.

Turning to FIG. 4, a hardware embodiment of the present invention is shown generally at 100, consisting of a base motherboard 102 and thirty-two daughter card connectors 104, the required power supply 106, an adder tree, 108, control, TGC, and data transmission circuits 110. A set of thirty-two daughter cards 112, each with eight ultrasonic pulsers/receivers “P”, eight analog-to-digital converters “A”, and eight “F” FPGAs are coupled to the motherboard daughter card connectors 104, with a second probe adapter motherboard 114 coupled to each daughter card to complete the system.

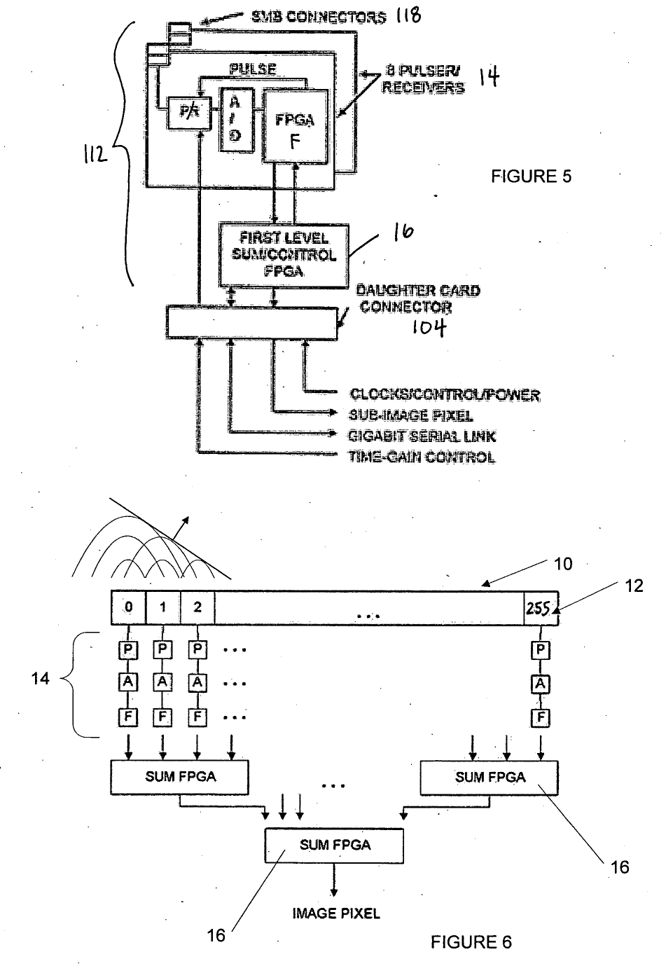

Each daughter card 112 is controlled via a control subsystem that generates 32 in-phase sample clocks and 32 in-phase image generation clocks, one for each daughter card, and the required “start acquisition” and “start image formation” control signals. One “start acquisition” and one “start image formation” control signal would be generated for each daughter card. Turning to FIG. 5, a block diagram of the daughter card 112 is shown. The “First Level Sum/Control FPGA” on each daughter card would perform two functions in this implementation: performing the first level sum during image generation, control signal, clock buffering, and distribution. The FPGA preferably uses internal delay locked loops to buffer and regenerate the sample and image generation clocks from the daughter card connectors 104 and to distribute them to the eight primary “F” FPGAs on the daughter card 112.

A high-speed serial link from the control subsystem to each daughter card 112 enables the parameters associated with each “G” block to be initialized, via a local host, by remote workstation. The contents of each block RAM 18 can be read or written, allowing received sample data to be sent to a remote host workstation for storage. Alternatively, sample data previously acquired can be loaded into the block RAMs 18 for processing. Preferably, eight coaxial SMB connectors 118 are utilized to link each daughter card 112 to the second “motherboard” 114 that implements a probe bulkhead adapter. SMB connectors are utilized since the male and female connectors can connect back-to-back and “snap” together. The use of the second “motherboard” 114 enables easy adaptation to a wide variety of array transducers.

There are alternative architectures that are modifications or variations of the architecture proposed above. For example, rather than use additional FPGAs to sum the sub images, this summation could be done in a serial fashion as described in U.S. Pat. No. 6,719,693 B2. There are many other architectures which are modifications of the architectures presented here that are extensions of the basic idea of accessing all block memories in parallel and adding them to first form sub-images and then adding the sub-images to form the final synthetic focus image. Use of the system for real-time synthetic focus imaging is described in more detail in U.S. Pat. No. 6,719,693 B2.

In the embodiments of the system described in U.S. Pat. No. 6,719,693 B2, the system cannot be used to form images using conventional sum beam-forming/focus-and-steer imaging techniques since only one transducer is pulsed at a time. By modifying the design so that multiple transducers can be pulsed in sequence with accurate time delays, the system can be used to form both synthetic focus images and sum beam-forming/focus-and-steer images.

In conventional sum beam-forming/focus-and-steer systems, several transducers in the array are pulsed each time an acquisition cycle is initiated. The transducers are pulsed in sequence, using accurate time delays, so as to create a transmit beam that propagates at a desired deflection angle relative to the transducer array. By changing the time delays from one acquisition cycle to the next, a sector scan can be created.

FIG. 6 illustrates the type of wave front that can be generated when a conventional system pulses multiple transducer array elements. In this simplified illustrative example, three elements have been pulsed: element 0 was pulsed first (so that energy from element 0 has propagated the farthest), then after a short delay element 1 was pulsed, and then after another delay element 2 was pulsed. Pulsing in this manner creates, effectively, a wave front that propagates at an angle relative to the array, as indicated by the arrow in FIG. 6. By changing the delays used, different deflection angles can be generated.

It is also possible to focus the transmitted energy at a point in the material being imaged. This requires different timing delays than those required in the example here, but which is a straight-forward extension of the same basic principle. This technique allows this type of system to do what is termed “transmit focus,” although only one point in the image is in true focus during each transmit cycle.

By adding the ability to pulse multiple transducers with accurate time delays, the architecture described in U.S. Pat. No. 6,719,693 B2 can support the transmit beam generation capability with transmit focus used in conventional sum beam-forming/focus-and-steer systems. This capability is preferably added by using a digitally controllable digital delay between each “F” FPGA and the “pulse” input to each pulser/receiver “P”. For example, a Dallas Semiconductor DS1023 integrated circuit provides step sizes as small as 0.5 ns, and with eight programming inputs, can be set to one of 256 discrete delays. The programming inputs to each DS1023 are controlled by the associated “F” FPGA to delay the pulse on the associated transducer element to achieve the desired time delay. Many other digitally controlled delay solutions are available.

For each transmit beam generated by a conventional system, ultrasound energy from each image point along the beam reflects back to all elements of the array with a time-of-flight based on the distance from the point to the transducer element. After pulsing, conventional systems perform receive focusing through the use of time delays to add the appropriate time-gain corrected receive signal from each transducer to form a “scan line” of ultrasound data, as described by Albert Macovski in “Medical Imaging Systems”, Prentice-Hall, Englewood Cliffs, N.J., 1983, herein incorporated by reference. This scan line represents one “ray” or “vector” in the ultrasound image, and digital systems require a scan conversion step to convert the data (which is in polar form) into Cartesian form for display on a raster scan monitor, as described by Richard and Arthur in “Real-Time Ultrasonic Scan Conversion via Linear Interpolation of Oversampled Vectors”, Ultrasonic Imaging, Vol. 16, pp 109-123, April 1994, incorporated herein by reference.

In the present invention, the architecture described in U.S. Pat. No. 6,719,693 B2 is adapted to performing receive focus when used to form real-time sum beam-forming/focus-and-steer images. When operating in sum beam-forming/focus-and-steer mode, the dual-port block RAMs 18 in the “F” FPGAs are filled, in turn, as transmit beams sweep out a sector scan. If a 60 degree scan is used, for example, the first block RAM 18 in each “F” FPGA is filled when the transmit beam is deflected +30 degrees from the line perpendicular to the transducer face, and the last block RAM 18 is filled when the transmit beam is deflected −30 degrees. For a given image, this would allow as many “scan lines” to be acquired as there are block RAMs 18 in the “F” FPGAs (assuming one “F” FPGA is assigned to each transducer). For the preferred embodiment described herein, 256 scan lines could be acquired.

When operating in sum beam-forming/focus-and-steer mode, instead of stepping through the image pixel-by-pixel and adding the appropriate samples from all of the block RAMs 18 to form each image pixel, as is done in synthetic focus mode, the “G” blocks step through the image “vector-by-vector” adding the appropriate samples from the block RAM 18 in each “F” FPGA associated with the vector being formed. In the example of the 60 degree system given just above, the “G” blocks associated with the first block RAM 18 in each “F” FPGA would generate the memory addresses required access the data samples associated with the first sample of the first (+30 degree) scan line, then the second data sample of the first scan line, and so on, until the first scan line had been generated. The remaining “G” blocks would not generate addresses during generation of the first scan line since the block RAMs 18 associated with these “G” blocks hold data associated with different scan lines. Block RAMs 18 not used to form a particular scan line would have to be designed to output a zero value when they were not being accessed. This could be done using special gating logic or by designing the system so that a zero value was always stored at address zero in each block RAM 18. In this case, the “G” blocks would be designed to output address zero until they are used to generate their associated scan line.

In this particular embodiment, the SUM FPGAs 16 sum the “sub-vectors” generated by the “F” FPGAs to produce the final scan line. After the first scan line is generated, the second scan line is generated in a similar manner, then the third scan line, until the entire set of scan lines required to form an image has been generated.

The scan lines associated with a complete image are preferably envelope detected and scan converted by a local host processor or by other means to form the final image.

When operating in sum beam-forming/focus-and-steer mode, the system optionally overlaps the pulse/acquisition cycle associated with one scan line with the computation of a different scan line. The fact that this can be done is one of the primary advantages of sum beam-forming/focus-and-steer imaging over synthetic focus imaging.

The fact that sum beam-forming/focus-and-steer imaging does not require the complete dataset implies that it is not necessary to use all of the block RAMs 18 in each “F” FPGA to form sum beam-forming/focus-and-steer images. Using just one block RAM 18 in each “F” FPGA in the embodiment described above, data for a single scan line can be acquired into a block RAM 18 and then the scan line generated from that block RAM 18, then the data for a second scan line acquired, etc. With two or more block RAMs 18, scan line acquisition and generation can overlap as described above. The “G” address generators will generate different addresses when used in this mode, but their fundamental design is the same. This mode of operation will have advantages in some physical implementations of the architecture.

It is not necessary to use the same block RAMs 18 and address generators “G” for both synthetic-focus imaging and sum beam-forming/focus-and-steer imaging. With appropriate multiplexing, completely independent subsystems can be contained in each “F” FPGA, and this embodiment will have advantages in some implementations of the architecture.

The system described here is therefore capable of supporting dual-use operation, in which it can support synthetic focus imaging via techniques described in U.S. Pat. No. 6,719,693 B2 and sum beam-forming/focus-and-steer imaging using techniques described above.

A dual-use system will allow the images formed using synthetic focus methods to be compared directly with images formed using sum beam-forming/focus-and-steer techniques, with both images formed using the same transducer and front end electronics. Using the same transducer and front end electronics is important for doing a fair comparison since synthetic focus imaging may result in a loss of penetration relative to that available using sum beam-forming/focus-and-steer techniques. With synthetic focus imaging, the highest acoustic intensity is near the transducer surface, while with sum beam-forming/focus-and-steer techniques it is at the transmit focal point. Thus, synthetic focus imaging will start with a signal-to-noise disadvantage that will be compensated for, to some degree, by the constructive interference that occurs during image formation. The noise floor of the front end electronics of a particular dual-mode system will determine the extent to which depth penetration is lost or gained.

In a clinical setting, a sonographer could image a beating heart using the system in sum beam-forming/focus-and-steer imaging mode, so as to avoid the motion artifacts associated with the long acquisition time associated with synthetic focus imaging, and then switch the system to synthetic focus imaging mode to image a static breast or prostate looking for tumors. In synthetic focus mode, the images produced could provide additional diagnostic information not available in sum beam-forming/focus-and-steer images. For reference and comparison purposes, while scanning a breast, for example, the sonographer could switch the system to sum beam-forming/focus-and-steer imaging mode and quickly view images of the same breast formed using sum beam-forming/focus-and-steer techniques.

It is also possible to use tissue properties extracted in one mode to improve the images produced by the other mode, e.g., speed of sound estimates extracted in synthetic focus mode can be used to improve the images produced using sum beam-forming/focus-and-steer techniques. Synthetic focus images can be first generated assuming a flat velocity map, with the velocity assumed to be equal everywhere to the average background velocity, and then the velocity map modified iteratively as part of an optimization step to obtain improved focus. The resulting velocity map, which represents the differing velocities at different points in the tissue, can then be used in sum beam-forming/focus-and-steer imaging mode to form more accurate images. While this technique will be more applicable to static targets, such as breast or prostate, it can also find utility when imaging the heart or other organs. The dual-use functionality of the architecture described here will, therefore, have important clinical advantages.

The present invention can be embodied in-part in the form of computer-implemented processes and apparatuses for practicing those processes. The present invention can also be embodied in-part in the form of computer program code containing instructions embodied in tangible media, such as floppy diskettes, CD-ROMs, hard drives, or an other computer readable storage medium, wherein, when the computer program code is loaded into, and executed by, an electronic device such as a computer, micro-processor or logic circuit, the device becomes an apparatus for practicing the invention.

The present invention can also be embodied in-part in the form of computer program code, for example, whether stored in a storage medium, loaded into and/or executed by a computer, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented in a general-purpose microprocessor, the computer program code segments configure the microprocessor to create specific logic circuits.

In view of the above, it will be seen that the several objects of the invention are achieved and other advantageous results are obtained. As various changes could be made in the above constructions without departing from the scope of the invention, it is intended that all matter contained in the above description or shown in the accompanying drawings shall be interpreted as illustrative and not in a limiting sense.

1. A dual-mode ultrasonic imaging system having a plurality of transmitter/receiver array elements configured to propagate at least one pulse of ultrasonic energy towards the surface of a sample, and to receive a return signal, the improvement comprising:
said array elements configured for selective operation in first imaging mode, said first imaging mode being a synthetic focus imaging mode; and
said array elements configured for selective operation in a second imaging mode, said second imaging mode being a sum beam-forming/focus-and-steer imaging mode.

2. The dual-mode ultrasonic imaging system of claim 1 wherein said first and second imaging modes generate substantially real-time images.

3. The dual-mode ultrasonic imaging system of claim 1 wherein each of said plurality of said transducer array elements is configured to pulse independently in said synthetic focus imaging mode.

4. The dual-mode ultrasonic imaging system of claim 3 configured to form synthetic focus images by summing select data samples from a complete dataset of ultrasonic return signals received at each transducer array element.

5. The dual-mode ultrasonic imaging system of claim 1 wherein a plurality of said transducer array elements are configured to pulse in sequence with predetermined time delays in said sum beam-forming/focus-and-steer imaging mode.

6. The dual-mode ultrasonic imaging system of claim 5 configured to form sum beam-forming/focus-and-steer images with receive focus by summing selected data samples from a collection of received scan line data.

7. The dual-mode ultrasonic imaging system of claim 1 configured to form enhanced synthetic focus images using sample properties extracted in a sum beam-forming/focus-and-steer imaging mode.

8. The dual-mode ultrasonic imaging system of claim 1 configured to form enhanced sum beam-forming/focus-and-steer images using sample properties extracted in a synthetic focus imaging mode.

9. A method for ultrasonic imaging including the steps of:
selecting between at least a synthetic focus imaging mode and a sum beam-forming/focus-and-steering imaging mode;
activating one or more ultrasonic transducers in an ultrasonic transducer array responsive to said selected imaging mode;
acquiring one or more ultrasonic return signals at said ultrasonic transducer array; and
generating an ultrasonic image utilizing said acquired one or more ultrasonic return signals.

10. The method for ultrasonic imaging of claim 9 wherein responsive to selecting said synthetic focus imaging mode, said activating step further includes activating one or more ultrasonic transducers in said ultrasonic transducer array independently.

11. The method for ultrasonic imaging of claim 9 wherein responsive to selecting said sum beam-forming/focus-and-steering imaging mode, said activating step further includes activating a plurality of said ultrasonic transducers in said ultrasonic transducer array in a predetermined time delayed sequence.

12. The method for ultrasonic imaging of claim 9 further including the step of utilizing sample data acquired in a first imaging mode to alter at least one imaging parameters of a second imaging mode.

13. The method for ultrasonic imaging of claim 9 further including the step of comparing images acquired in said synthetic focus imaging mode with images acquired in said sum beam-forming/focus-and-steering imaging mode.

14. An improved ultrasonic imaging system having a plurality of transmitter/receiver array elements configured to propagate pulses of ultrasonic energy towards the surface of a sample, and to receive return echoes, the improvement comprising:
a plurality of processing circuit sets, each of said plurality of processing circuit sets associated with one of the plurality of transmitter/receiver array elements;
an associated pulse signal delay means operatively coupled to each of said plurality of transmitter/receiver array elements;
said pulse signal delay means configured for selective operation in first imaging mode, said first imaging mode being a synthetic focus imaging mode; and
said pulse signal delay means configured for selective operation in a second imaging mode, said second imaging mode being a sum beam-forming/focus-and-steer imaging mode;
wherein each of said plurality of processing circuit sets is configured to store at least one data value representative of a digitized ultrasonic return echo received at an associated transmitter/receiver array element; and
wherein each of said plurality of processing circuit sets is further configured for parallel access to said stored data values during an image generation mode.

15. The improved ultrasonic imaging system of claim 14 further including at least one summing circuit configured to receive input from each of said plurality of processing circuit sets representative of a component of an ultrasonic image pixel, and to generate an output representative of an ultrasonic image pixel.
